(19) United States
(12) Patent Application Publication
(10) Pub. No.: US 2009/0177040 A1
(43) Pub. Date: Jul. 9, 2009

(54) Instrument shaft

(75) Inventors: Edwin Lyons, Galway (GB); Dougles J. Rose-Innes, Gwent (GB)

Correspondence Address: NIXON & VANDERHYE, PC, 901 NORTH GLEBE ROAD, 11TH FLOOR, ARLINGTON, VA, 22203, US

(73) Assignee: Gyrus Medical Limited, Cardill (GB)

(21) Appl. No.: 12/314,522

(22) Filed: Dec. 11, 2008

Related U.S. Application Data
(60) Provisional application No. 61/006,710, filed on Jan. 28, 2008.

(30) Foreign Application Priority Data
Jan. 4, 2008 (GB) 0800159.6

(51) Int. Cl. A61B 1/008

(52) U.S. Cl. 600/141

(57) ABSTRACT

A shaft for a flexible endoscopic instrument is provided in which the shaft comprises a generally tubular frame member, the tubular frame member providing the shaft with at least one flexible section along the length of the shaft, the flexible section having a greater flexibility than at least one other section of the shaft. The flexible section of the shaft has a proximal end and a distal end, and is provided with first and second series of slots, the slots of the first series alternating with the slots of the second series to form an offset pattern of staggered slots in the frame member to provide the different stiffness properties. Each of the slots in a selected one of either the first or second series of slots is of a different length as compared to other slots in that series of slots, the slots in the selected series varying in length according to a predetermined progression, such that the flexible section is designed to deflect in a controlled and predetermined manner.

### CROSS-REFERENCE TO RELATED APPLICATION

This application claims the benefit of Provisional Application No. 61/006,710, filed Jan. 28, 2008, the entire contents of which are hereby incorporated by reference in this application.

### FIELD OF THE INVENTION

The present invention relates to a shaft for an endoscopic instrument such as an endoscope or electrosurgical instrument for use in the treatment of tissue.

### BACKGROUND OF THE INVENTION

U.K. Patent Application No. 2130885 discloses a flexible distal end portion for an endoscope. The end portion is made from a plastics material with vertebrae connected by an elongate member or spine. U.S. Pat. No. 5,938,588 discloses an endoscope with wire sheaths made as solid tubes from a superelastic alloy material. U.S. Pat. No. 6,749,560 discloses an endoscope shaft which is provided with a region having an offset pattern of staggered slots. The present invention attempts to provide an improvement over these prior art devices.

### SUMMARY OF THE INVENTION

Accordingly, there is provided a shaft for a flexible endoscopic instrument, the shaft comprising a generally tubular frame member, the tubular frame member providing the shaft with at least one flexible section along the length of the shaft, the flexible section having a greater flexibility than at least one other section of the shaft, wherein the or each flexible section of the shaft has a proximal end and a distal end, and is provided with first and second series of slots, the slots of the first series alternating with the slots of the second series to form an offset pattern of staggered slots in the frame member to provide the different stiffness properties, wherein each of the slots in a selected one of either the first or second series of slots is of a different length as compared to adjacent slots in that series of slots, the slots in the selected series varying in length according to a predetermined progression, such that the flexible section is designed to deflect in a controlled and predetermined manner.

By providing slots of a different length, the shaft has a flexible section in which the flexing of the shaft is preferentially controlled, i.e. the curving of the shaft will take place in a known and controlled manner. The slots in the selected series conveniently vary in length such that the ends of the slots in the series on one side of the shaft form a straight line over some or all of the flexible section. Conveniently, the straight line formed by the ends of the slots runs at a predetermined angle to the longitudinal axis of the shaft. According to one arrangement, the length of the slots increases towards the distal end of the flexible section.

In one convenient arrangement, the flexible section comprises two or more regions with a transition point therebetween, and the length of the slots in the selected series varies such that the ends of the slots in the series on one side of the shaft form a straight line at a first predetermined angle to the longitudinal axis of the shaft in the first region, and a second different predetermined angle to the longitudinal axis of the shaft in the second region.

Preferably, the length of the slots in the first region changes in a first sense, and the length of the slots in the second region changes in an opposite sense.

Conveniently, the length of the slots increases from each end of the flexible section, so as to be its greatest at the transition point. In this way, the flexibility of the flexible section can be varied along its length, typically so as to be most flexible at the transition point, and stiffer towards each end of the flexible section.

Conveniently, each of the slots in both the first and second series of slots is of a different length as compared to adjacent slots in that series of slots. Typically, the straight line formed by the ends of the slots runs at a predetermined angle to the longitudinal axis of the shaft, and also from each other such that the straight line formed by the ends of the slots in the first series runs at a predetermined angle from the straight line formed by the ends of the slots in the second series. The angle between the two straight lines can be chosen depending on the stiffness required for the flexible section, and also for the preferential deflection desired. Conveniently, the predetermined angle is between 0.5 and 6 degrees, typically between 1 and 3 degrees, and preferably between 1.5 and 2 degrees.

In one convenient arrangement, the flexible section comprises two or more regions with a transition point therebetween, and the length of the slots in one or both series of slots varies such that the ends of the slots form a straight line at a first predetermined angle to the longitudinal axis of the shaft in the first region, and a second predetermined angle to the longitudinal axis of the shaft in the second region. Therefore, the flexible section could comprise two, three or even more regions, with the slots in each region forming a straight line each with a different predetermined angle to the longitudinal axis of the shaft. As before, the length of the slots in the first region typically changes in a first sense, and the length of the slots in the second region changes in an opposite sense. Once again, the length of the slots conveniently increases from each end of the flexible section, so as to be its greatest at the transition point.

In one convenient arrangement, at least one slot in the first series towards the distal end of the flexible section is of a sufficient length to overlap with at least one slot in the second series of slots. Overlapping slots creates a more flexible characteristic to that part of the flexible section. Conversely, at least one slot in the first series towards the proximal end of the flexible section is of a sufficient length so that it does not overlap with any of the slots in the second series of slots. If the slots do not overlap, that part of the flexible region is stiffer and more resistant to curvature. Conveniently, the slots are such that they overlap towards the distal end of the region, but do not overlap towards the proximal end of the region, creating a flexible region which is more flexible at its distal end, and less so at its proximal end.

In one arrangement, the predetermined progression is such that the flexible section deflects evenly throughout its length. This helps to reduce strain on any single part of the flexible section, and increase its working life. Alternatively, the predetermined progression is such that the flexible section deflects progressively from one end to the other. Conveniently, the predetermined progression is such that the flexible section deflects progressively, starting from its distal end and progressing towards its proximal end. In this way, when the flexible section is urged to bend, the bending of the section will be initiated its distal end, as opposed its proximal end, or at some unknown other point in between. The shaft can, therefore, be designed to curve in a controlled manner, starting with a deflection towards the distal end of the flexible section, and continuing to deflect with the curvature progressing proximally along the flexible section until the whole of the flexible region is in a curved condition.

Alternatively, the predetermined progression is such that the flexible section deflects progressively, starting from its proximal end and progressing towards its distal end. Whichever controlled deflection is desired, a controlled deflection can be achieved by an appropriate selection of the predetermined progression, and in particular the angle between the straight lines formed by the ends of the slots of each set.

In one convenient arrangement, the frame member comprises a tube wall defining a central channel. This is typically employed where the shaft is used in an endoscope, or in a surgical instrument with a central lumen for the passage of fluid, for suction, or for containing electrosurgical leads or other components. The frame member conveniently has a substantially uniform outer dimension along substantially the entire length of the shaft, and the tube wall typically has a substantially uniform tube wall thickness and a substantially uniform outer diameter. Preferably, the slots extend into the tube wall a distance about two-thirds or less than the outer diameter of the tube wall.

As mentioned previously, the shaft can be employed in a surgical instrument such as an endoscope or tissue treatment instrument. The surgical instrument conveniently further comprises a control section, and an active deflection control wires connected to the control section. The deflection control wires can be used to cause the deflection of the flexible region of the shaft. Conceivably, the control wires could be provided with sheaths, or alternatively the control wires could be in the form of individual wires twisted together to form a cable.

### BRIEF DESCRIPTION OF THE DRAWINGS

The invention will now be described in more detail, by way of example only, with reference to the accompanying drawings, in which:

FIG. 1 is a schematic perspective view of an endoscopic shaft according to the invention;

FIG. 2 is a schematic side view of the shaft of FIG. 1;

FIG. 3 is a schematic plan view of the shaft of FIG. 1;

FIG. 4 is an enlarged view of the area marked A in FIG. 2;

FIG. 5 is a schematic perspective view of an alternative embodiment of an endoscopic shaft according to the invention;

FIG. 6 is a schematic side view of the shaft of FIG. 5;

FIG. 7 is a schematic plan view of the shaft of FIG. 5;

FIG. 8 is an enlarged view of the area marked A in FIG. 6;

FIG. 9 is a schematic perspective view of a further alternative embodiment of an endoscopic shaft according to the invention;

FIG. 10 is a schematic side view of the shaft of FIG. 9;

FIG. 11 is a schematic plan view of the shaft of FIG. 9;

FIG. 12 is an enlarged view of the area marked A in FIG. 10;

FIGS. 13 to 18 are schematic side views of various embodiments of endoscopic shafts according the present invention, shown subject to a deflection force, and

FIGS. 19 to 22 are schematic side views of alternative embodiments of endoscopic shaft according to the invention.

### DESCRIPTION OF PREFERRED EMBODIMENTS

Referring to FIGS. 1 to 4, a shaft for an endoscopic instrument is shown generally at 1, and includes a flexible section 2 having a distal end 3 and a proximal end 4. The flexible section 2 includes a first set of slots 5 and a second set of slots 6. The slots 5 and 6 are cut into the shaft 1, each slot extending approximately half way around the circumference of the shaft. The slots 5 and 6 are parallel to one another, and extend substantially at 90 degrees to the longitudinal axis of the shaft. The first set of slots 5 are cut into the upper surface of the shaft 1, while the second set of slots 6 are cut into the lower surface of the shaft, with the slots 5 being interleaved with the slots 6. The slots 5 and 6 are typically cut out of the shaft 1 with a laser (not shown), or can alternatively be formed by a wire erosion process.

Referring to the upper set of slots 5, the length of the slots varies along the flexible section 2, such that the slot 5a at the distal end 3 of the flexible section is slightly longer than the adjacent slot 5b. In similar fashion, the slot 5b is slightly longer than the next slot 5c, and so on such that the length of the slots 5 progressively decreases from the distal end 3 to the proximal end 4. The slot 5p at the proximal end of the flexible section 2 is the shortest of the slots 5.

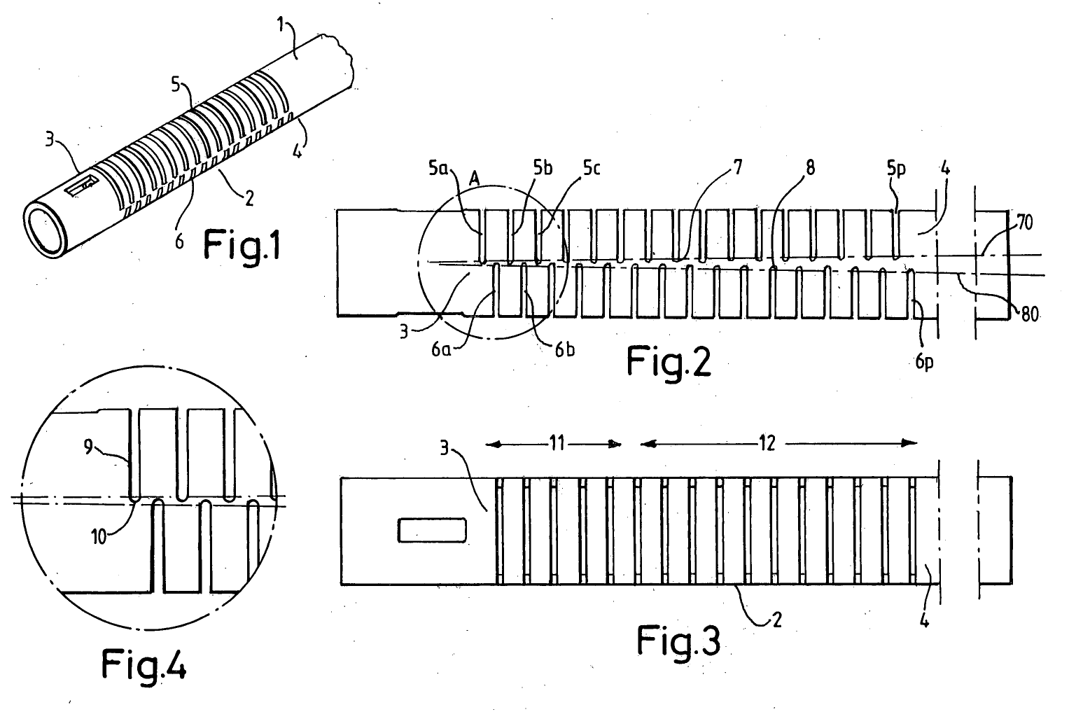

The same arrangement is present with respect to the lower set of slots 6, with the slot 6a at the distal end 3 of the flexible section 2 being slightly longer than slot 6b etc, and slot 6p at the proximal end 4 being the shortest slot of the set 6. The change in length of the slots 5 and 6 is constant as between adjacent slots, such that the ends 7 of the upper slots 5 and the ends 8 of the lower slots 6 form two straight lines 70 and 80 respectively. The lines 70 and 80 diverge from one another, as well as from the longitudinal axis of the shaft 1. In FIGS. 1 to 4, the lines 70 and 80 diverge from one another at an angle of approximately 1.5 degrees. As shown in the plan view of FIG. 3, the shaft 1 is radially symmetrical, such that the slots 5 and 6 would appear identical to FIGS. 1 & 2 if viewed from the reverse side.

FIG. 4 shows an enlarged portion of FIG. 2, from which it can be seen that each of the slots 5 and 6 has parallel sides 9 with a generally semicircular end portion 10. The ends 7 and 8 of the slots 5 and 6 can be taken from the tip of the semicircular end portion, or alternately from the ends of the parallel sides. In FIGS. 1 to 4, the ends 7 and 8 are taken from the ends of the parallel sides 9, as can be seen most clearly from FIG. 4.

Owing to the variation in the lengths of the slots 5 and 6, the flexible section 2 has a first portion 11 in which the slots 5 and 6 are sufficiently long such that their ends overlap one with the other. This overlapping portion 11 is located towards the distal end 3 of the flexible section 2. Similarly, the flexible section 2 has a second portion 12 in which the slots 5 and 6 are not sufficiently long for their respective ends to overlap. This non-overlapping portion 12 is located towards the proximal end 4 of the flexible section 2. In FIG. 2, it can be clearly seen that the non-overlapping portion 12 is greater in length that the overlapping portion 11.

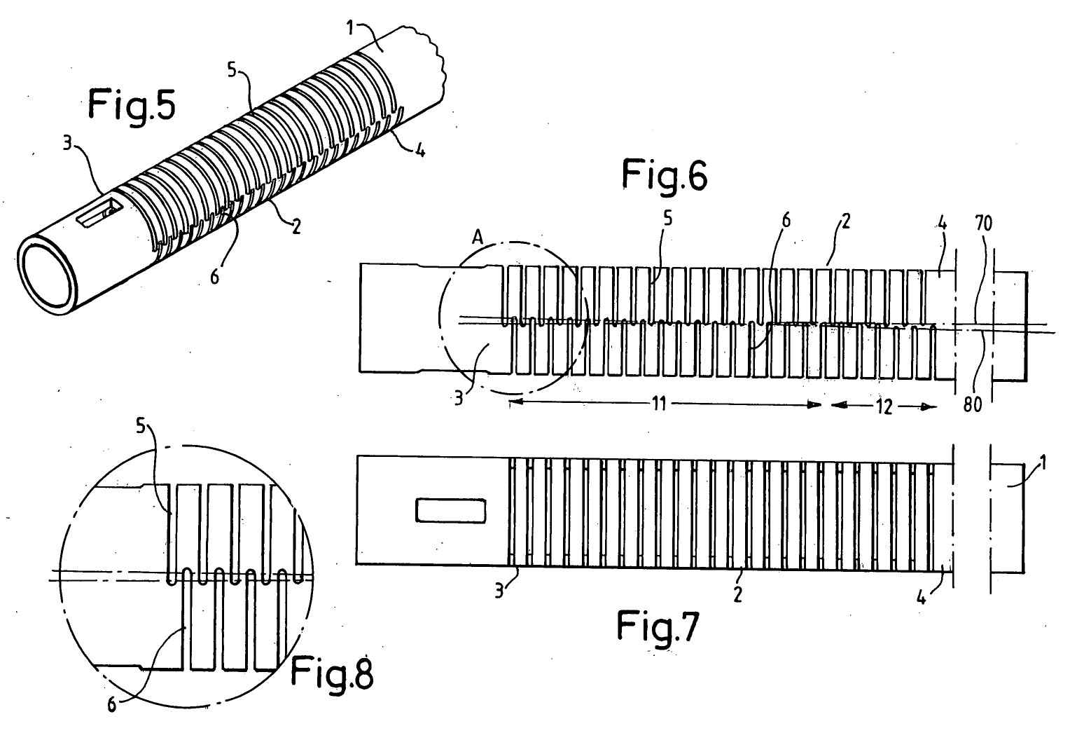

FIGS. 5 to 8 show an alternative embodiment of the shaft 1, in which many of the features are similar to those of FIGS. 1 to 4 and have been designated with like reference numerals. The flexible section 2 of this embodiment is provided with a greater number of slots 5 and 6, located more closely together. The slots 5 and 6 are still arranged such that the lines 70 and 80 diverge from one another at an angle of 1.5 degrees, but the slots are generally longer such that the overlapping portion 11 is of much greater length than the non-overlapping portion 12. The flexible section 2 of the embodiment of FIGS. 5 to 8 is accordingly designed to be much more flexible as compared to the relatively more rigid flexible section of the embodiment of FIGS. 1 to 4.

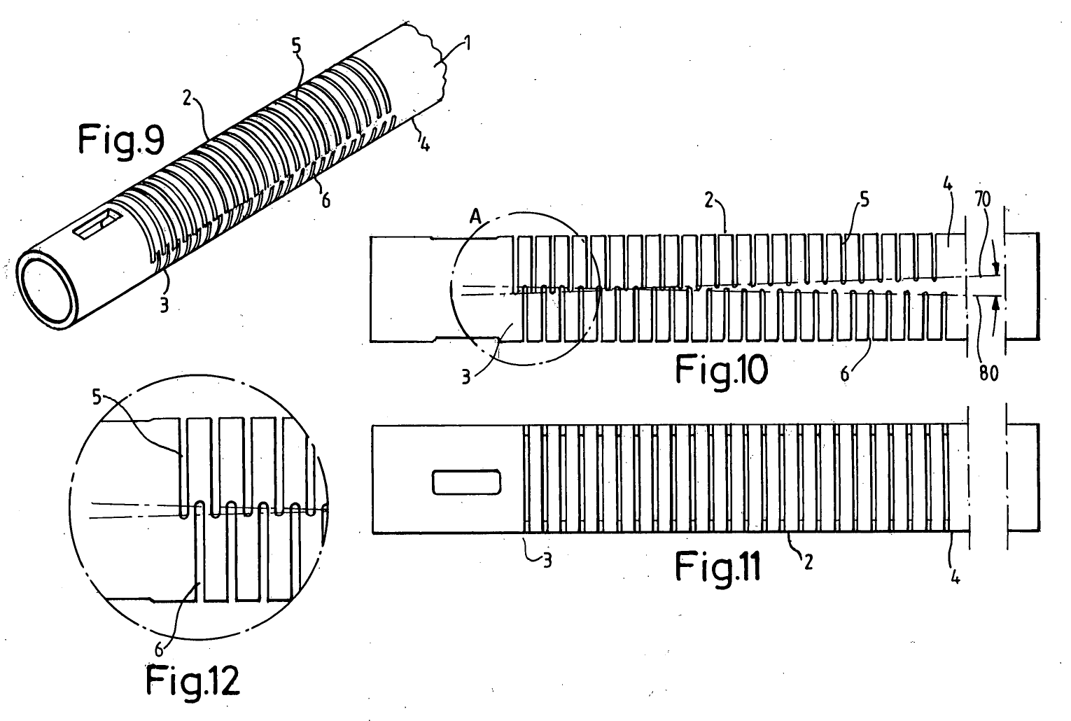

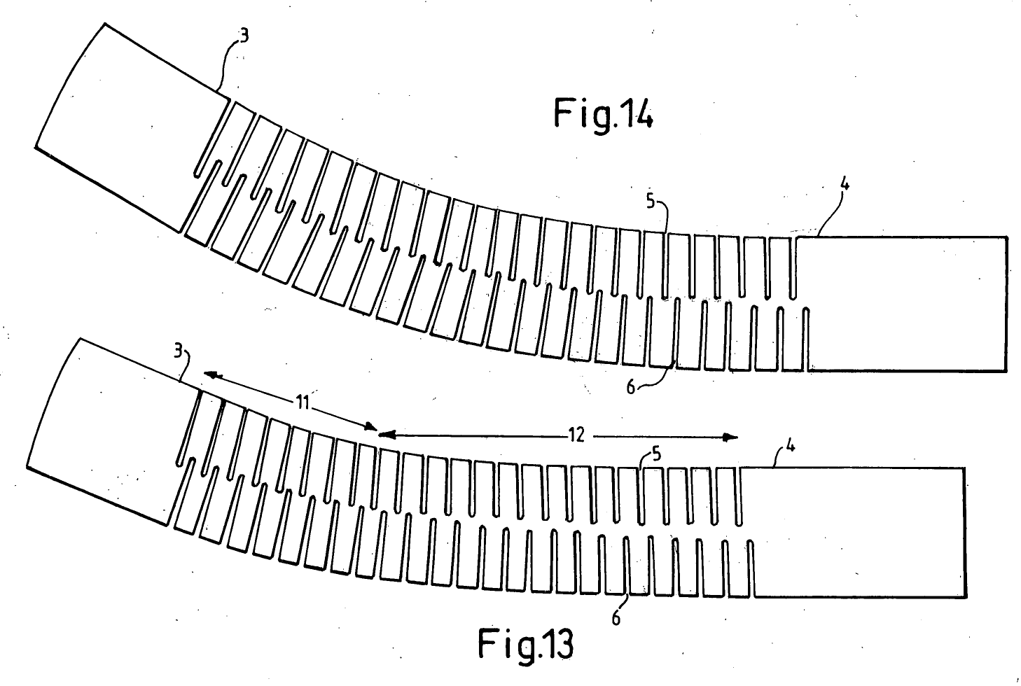

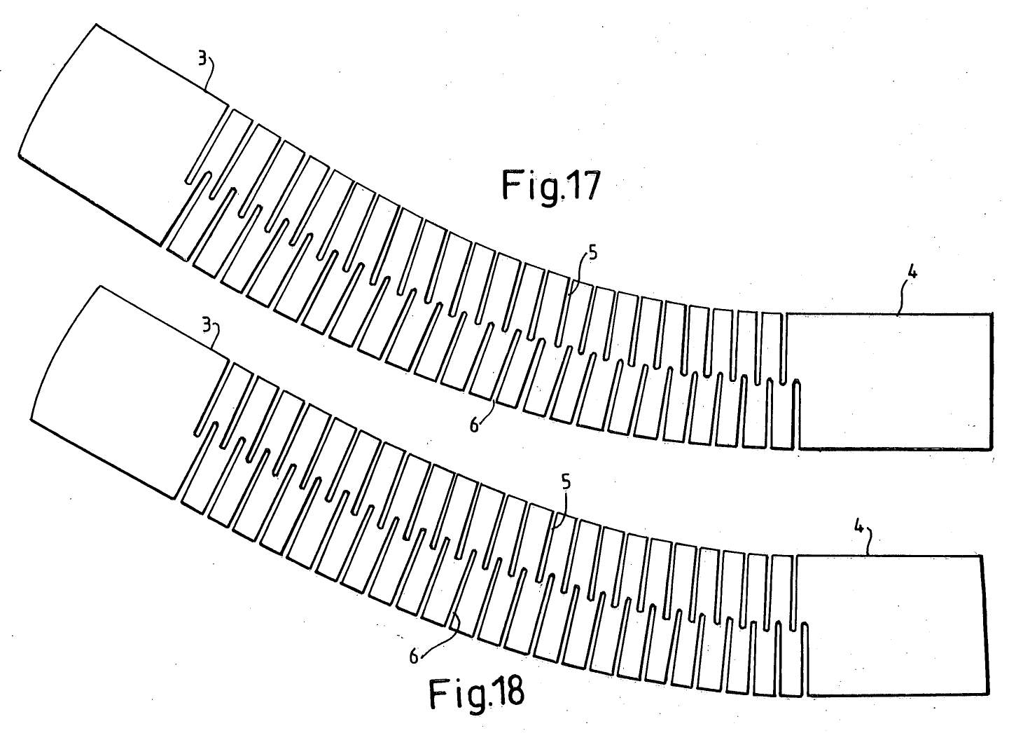

FIGS. 9 to 12 show a further embodiment in which the number and spacing of the slots 5 and 6 are similar to those of FIGS. 5 to 8. However, in this embodiment, the change in length of the slots 5 and 6 is more rapid, resulting in the lines 70 and 80 diverging from one another at an angle of 3 degrees. In this embodiment, the overlapping and non-overlapping portions 11 and 12 are of about equal length. In this embodiment, a flexible section 2 is created in which the flexibility is much greater at the distal end 3 as compared with the proximal end 4. The varying effects of different diverging angles will now be illustrated with reference to FIGS. 13 to 18.

FIG. 13 shows a shaft 1 similar to that of FIGS. 9 to 12, having slots 5 and 6 that vary in length such that their ends form straight lines 70 & 80 diverging at an angle of 3 degrees. When the shaft 1 is subjected to a deflection force, for example by the manipulation of active deflection wires (not shown), the flexible section 2 starts to bend. Owing to the change in the length of the slots 5 and 6, and the 3-degree angle between the lines 70 & 80, this deflection will begin primarily at the distal end 3 of the flexible section 2. The deflection will be constrained primarily within the overlapping portion 11, with the non-overlapping portion 12 remaining substantially straight, at least until a further deflection force is applied to the shaft 1. As a further deflection force is applied to the shaft 1, the flexible section 2 will deflect further, with the bending zone extending proximally along the flexible section towards the proximal end 4 thereof.

In contrast, FIG. 18 shows a shaft 1 having slots 5 and 6 that vary in length such that their ends form straight lines 70 & 80 diverging at an angle of only 0.5 degrees. When the shaft 1 is subjected to a deflection force, the flexible section 2 will bend with the deflection occurring primarily at the proximal end 4 of the flexible section. Unlike the embodiment of FIG. 13, the distal ends of the flexible section 2 remains essentially straight, with a further deflection force causing the bending zone to extend distally along the flexible section towards the distal end thereof.

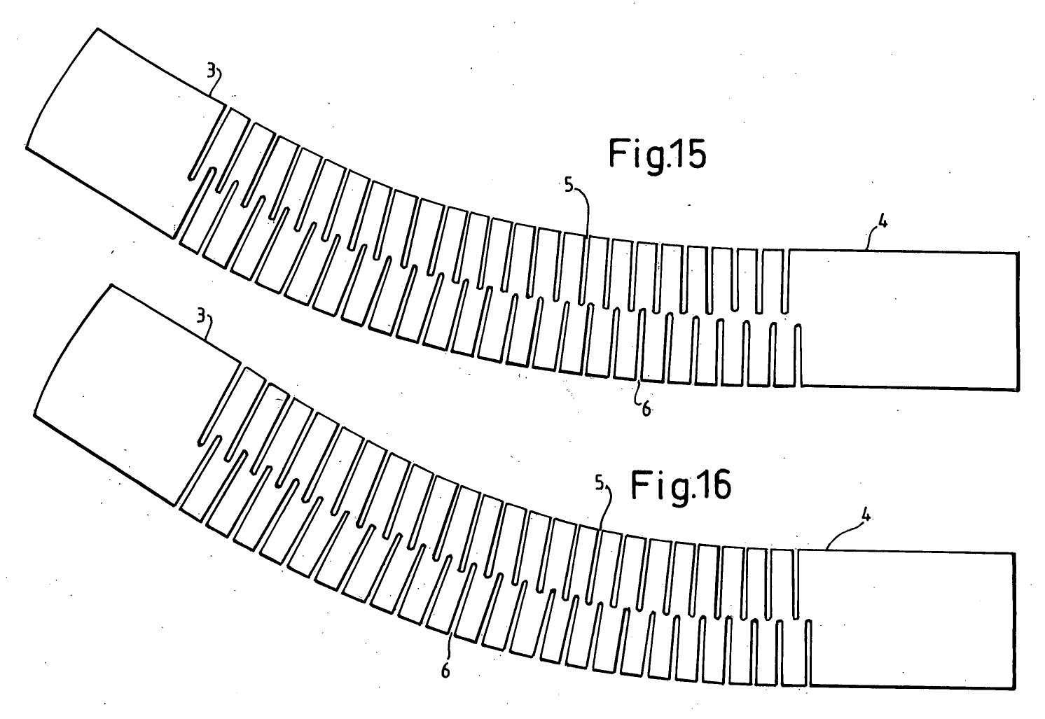

In between these two arrangements, FIG. 16 shows a shaft 1 similar to that of FIGS. 5 to 8, having slots 5 and 6 that vary in length such that their ends form straight lines 70 & 80 diverging at an angle of 1.5 degrees. When this shaft 1 is subjected to a deflection force, the bending that will occur will be distributed along the flexible section 2, starting towards the centre of the flexible section and extending both distally and proximally therefrom. FIG. 15 shows an embodiment in which the diverging angle is approximately 2 degrees, in which the bending is evenly distributed along the whole of the flexible section 2. Such an arrangement has the advantage that all areas of the flexible section 2 are stressed uniformly, and so this should help to ensure that no fatigue to the flexible section develops due to the flexible section always deflecting in the same area or areas.

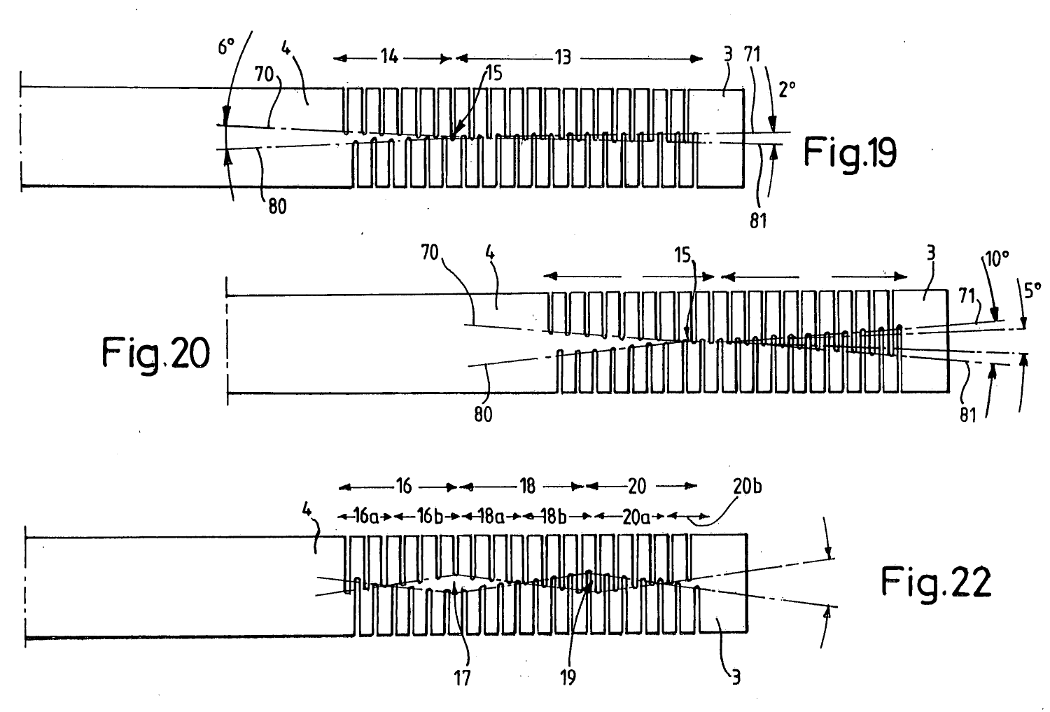

FIG. 19 shows a shaft 1 with a flexible section 2 having two regions 13 and 14, and a transition point 15 therebetween. The region 13 runs from the distal end 3 of the flexible section 2 to the transition point 15, and the region 14 runs from the transition point to the proximal end 4 of the flexible section. In the region 14, the slots 5 and 6 vary in length in a first sense, such that the slots increase in length from the proximal end 4 to the transition point 15. The ends of the slots 5 and 6 in the region 14 form two straight lines 70 and 80, and the ends of the slots in the region 13 form two straight lines 71 and 81. The straight lines 70 and 80 converge towards the transition point 15 at an angle of approximately 6 degrees. After the transition point 15, the slots 5 and 6 still continue to vary in length in the same sense, i.e. increasing in length as one moves distally along the flexible section 2. However, in the region 13, the straight lines 71 and 81 diverge from the transition point 15 at an angle of only 2 degrees.

In FIG. 19 the slots 5 and 6 vary in length such that the lines 70, 80 in the region 14 and 71, 81 in the region 13 are straight lines. FIG. 20 shows an alternative arrangement in which the slot length varies according to an increasing progression, such that the lines 70, 80, in the region 14 and the lines 71, 81, in the region 13 are all curved. Thus, the slots 5 and 6 converge, starting at the proximal end 4 at an angle of 10 degrees, which reduces steadily over the region 14 until it is approximately 5 degrees as the transition point 15 is reached. Similarly, the slots 5 and 6 continue to converge after the transition point 15, starting at an angle of 5 degrees near the transition point and increasing steadily over the region 13 until an angle of 10 degrees is reached at the distal end 3 of the flexible section 2. This gradual change in the angle of convergence further helps to control the flexing of the flexible section 2 in a controlled and repeatable manner.

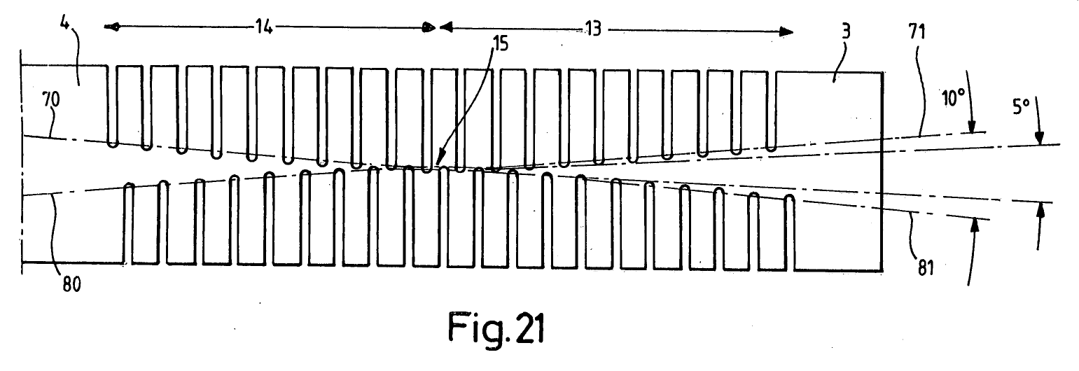

FIG. 21 shows a further embodiment in which the length of the slots 5 and 6 changes in accordance with an increasing and then decreasing progression. In FIG. 21, the length of the slots 5 and 6 increases from both ends of the flexible section 2 towards the transition point 15. Thus, the slots in the region 14 vary in length in a first sense, increasing in length between the proximal end 4 and the transition point 15. After the transition point 15, the slots 5 and 6 vary in length in an opposite sense, in that they decrease in length between the transition point 15 and the distal end 3. The angle between the lines 70, 80 in the region 14, and the lines 71, 81 in the region 13, varies between 5 degrees adjacent to the transition point 15, increasing to 10 degrees at each end of the flexible section 2.

Finally, FIG. 22 shows an embodiment in which there are multiple regions 16, 18 and 20 in which the length of the slots 5 and 6 varies in difference senses. Staring from the proximal end 4, the slots 5 and 6 decrease in length in the region 16, overlapping in a region 16a but not overlapping in a region 16b. At a transition point 17, the slots S and 6 start to increase in length in the region 18, not overlapping in a region 18a but overlapping once again in a region 18b. There then follows a further transition point 19, after which the slots 5 and 6 decrease in length once again in the region 20, overlapping in a region 20a but not overlapping in a region 20b, until the distal end 3 of the flexible section 2 is reached.

As will be seen from the above examples, various combinations of increasing and/or decreasing slot length can be employed, with different angles of divergence and convergence being present between different transition points. The transition points can be coincident with the points at which the slots start to overlap, or can be located at other regions along the flexible section 2.

The shaft 1 can be employed in a flexible endoscope, or alternatively in a flexible tissue-treatment instrument. Those skilled in the art will be familiar with both such situations, as well as the requirements for deflection capability associated with different instruments and procedures. In accordance with the present invention, the deflection characteristics of each shaft can be designed in a controlled and predetermined manner.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A shaft for a flexible endoscopic instrument, the shaft comprising a generally tubular frame member, the tubular frame member providing the shaft with at least one flexible section along the length of the shaft, the flexible section having a greater flexibility than at least one other section of the shaft, wherein the or each flexible section of the shaft has a proximal end and a distal end, and is provided with first and second series of slots, the slots of the first series alternating with the slots of the second series to form an offset pattern of staggered slots in the frame member to provide different stiffness properties, wherein at least some of the slots in a selected one of either the first or second series of slots are of a different length as compared to adjacent slots in that series of slots, the slots in the selected series varying in length according to a predetermined progression, such that the flexible section is designed to deflect in a controlled and predetermined manner.

2. The shaft according to claim 1, wherein the length of the slots in the selected series varies such that the ends of the slots in the series on one side of the shaft form a straight line over some or all of the flexible section.

3. The shaft according to claim 2, wherein the straight line formed by the ends of said slots runs at a predetermined angle to the longitudinal axis of the shaft.

4. The shaft according to claim 3, wherein the length of the slots increases towards the distal end of the flexible section.

5. The shaft according to claim 3, wherein the flexible section comprises two or more regions with a transition point therebetween, and wherein the length of the slots in the selected series varies such that the ends of the slots in the series on one side of the shaft form a straight line at a first predetermined angle to the longitudinal axis of the shaft in the first region, and a second different predetermined angle to the longitudinal axis of the shaft in the second region.

6. The shaft according to claim 5, wherein the length of the slots in the first region changes in a first sense, and the length of the slots in the second region changes in an opposite sense.

7. The shaft according to claim 6, wherein the length of the slots increases from each end of the flexible section, so as to be its greatest at the transition point.

8. The shaft according to claim 2, wherein each of the slots in both the first and second series of slots is of a different length as compared to adjacent slots in that series of slots.

9. The shaft according to claim 8, wherein the length of the slots in both series varies such that the ends of the slots in each series on one side of the shaft form a straight line over some or all of the flexible section.

10. The shaft according to claim 9, wherein the straight line formed by the ends of said slots runs at a predetermined angle to the longitudinal axis of the shaft.

11. The shaft according to claim 10, wherein the straight line formed by the ends of the slots in the first series runs at a predetermined angle from the straight line formed by the ends of the slots in the second series.

12. The shaft according to claim 11, wherein the flexible section comprises two or more regions with a transition point therebetween, and wherein the length of the slots in both series varies such that the ends of the slots in the series form a straight line at a first predetermined angle to the longitudinal axis of the shaft in the first region, and a second different predetermined angle to the longitudinal axis of the shaft in the second region.

13. The shaft according to claim 12, wherein the length of the slots in the first region changes in a first sense, and the length of the slots in the second region changes in an opposite sense.

14. The shaft according to claim 13, wherein the length of the slots increases from each end of the flexible section, so as to be its greatest at the transition point.

15. The shaft according to claim 1, wherein at least one slot in the first series towards the distal end of the flexible section is of a sufficient length to overlap with at least one slot in the second series of slots.

16. The shaft according to claim 1, wherein at least one slot in the first series towards the proximal end of the flexible section is of a sufficient length so that it does not overlap with any of the slots in the second series of slots.

17. The shaft according to claim 15, wherein the slots are such that they overlap towards the distal end of the flexible section, but do not overlap towards the proximal end of the flexible section.

18. The shaft according to claim 1, wherein the predetermined progression is such that the flexible section deflects evenly throughout its length.

19. The shaft according to claim 1, wherein the predetermined progression is such that the flexible section deflects progressively from one end to the other.

20. The shaft according to claim 19, wherein the predetermined progression is such that the flexible section deflects progressively, starting from its distal end and progressing towards its proximal end.

21. The shaft according to claim 19, wherein the predetermined progression is such that the flexible section deflects progressively, starting from its proximal end and progressing towards its distal end.

22. The shaft according to claim 1, wherein the frame member comprises a tube wall defining a central channel.

23. The shaft according to claim 22, wherein the tube wall has a substantially uniform tube wall thickness and a substantially uniform outer diameter.

24. The shaft according to claim 1, wherein the frame member has a substantially uniform outer dimension along substantially the entire length of the shaft.

25. A surgical instrument including a shaft, the shaft comprising a generally tubular frame member, the tubular frame member providing the shaft with at least one flexible section along the length of the shaft, the flexible section having a greater flexibility than at least one other section of the shaft, wherein the or each flexible section of the shaft has a proximal end and a distal end, and is provided with first and second series of slots, the slots of the first series alternating with the slots of the second series to form an offset pattern of staggered slots in the frame member to provide different stiffness properties, wherein at least some of the slots in a selected one of either the first or second series of slots are of a different length as compared to adjacent slots in that series of slots, the slots in the selected series varying in length according to a predetermined progression, such that the flexible section is designed to deflect in a controlled and predetermined manner.

26. The instrument according to claim 25, further comprising a control section, and active deflection control wires connected to the control section.

27. The instrument according to claim 25, wherein the surgical instrument is a tissue treatment instrument.

28. The instrument according to claim 25, wherein the surgical instrument is an endoscope.
